(19) United States
(12) Patent Application Publication
(10) Pub. No.: US 2009/0045082 A1
(43) Pub. Date: Feb. 19, 2009

(54) PACKAGING DEVICE FOR BIOLOGICAL PRODUCTS

(75) Inventors: Frederic Malboze, Quincy Voisins (FR); Philippe Hernandez, Vincennes (FR); Edward Mitchell, Paris (FR); Eric Sermet, Hangzhou (CN)

Correspondence Address: AIR LIQUIDE;Intellectual Property, 2700 POST OAK BOULEVARD, SUITE 1800, HOUSTON, TX, 77056, US

(73) Assignee: L'Air liquide Societe Anonyme Pour L'Etude Et L'Exploitation Des Procedes Georges Claude, Paris (FR)

(21) Appl. No.: 12/174,241

(22) Filed: Jul. 16, 2008

Related U.S. Application Data
(60) Provisional application No. 60/953,899, filed on Aug. 3, 2007.

(30) Foreign Application Priority Data
Jul. 20, 2007 (FR) 07-56648

(51) Int. Cl. A61B 19/02

(52) U.S. Cl. 206/223

(57) ABSTRACT

A packaging device for biological products, comprising a parallelepiped-shaped container having an upper face, a lower face, and two side faces extending in a longitudinal direction between two end faces and defining at least one first recess, the device comprising a distinct storage element to be housed immovably in the first recess of the container in a direction parallel to the longitudinal direction, the storage element comprising a storage zone for a first type of receptacle of biological products, the storage element and/or the container being conformed to bound at least one second recess for a second type of receptacle of biological products, the container comprising an opening on an end face provided for introducing or removing the storage element and/or the second type of receptacle, the device comprising a cover distinct from the container for selectively closing the opening.

This application claims the benefit of priority under 35 U.S.C. § 119 (a), (b) and (e) to French Application No. 07556648, filed Jul. 20, 2007, and U.S. Provisional Patent Application No. 60/953,899, filed Aug. 3, 2007, the entire contents of which are incorporated herein by reference.

### BACKGROUND

1. Field of the Invention

The present invention relates to a packaging device for biological products.

2. Related Art

In the context of samplings of biological products of a patient or of a donor, the biological products sampled are often stored in various receptacles, such as sampling bags, tubes or straws.

Data about the patient or the donor and about the member of the medical staff who took the sampling must be opposed on each receptacle by the person or by another member of the medical staff.

This apposition of data on the various receptacles is a tedious task for the medical staff and significantly increases the operating time. Furthermore, due to the large number of receptacles, the risks of error in the data opposed on them are not negligible.

It should be observed that these various receptacles are stored in various locations, thereby complicating their localization when medical tests have to be conducted on the biological products contained therein and also generating a large storage volume.

It is the object of the present invention to remedy all or part of these drawbacks, and consists advantageously in providing a packaging device for biological products which has a simple and economical structure, while facilitating the packaging of these biological products and decreasing the storage volume thereof.

### SUMMARY OF THE INVENTION

For this purpose, the invention relates to a packaging device for biological products, comprising a parallelepiped-shaped container having an upper face, a lower face, and two side faces extending in a longitudinal direction between two end faces and defining at least one first recess, the device comprising a distinct storage element to be housed immovably in the first recess of the container in a direction parallel to the longitudinal direction, the storage element comprising a storage zone for a first type of receptacle of biological products, the storage element and/or the container being conformed to bound at least one second recess for a second type of receptacle of biological products, the container comprising an opening on an end face provided for introducing or removing the storage element and/or the second type of receptacle, the device comprising a cover distinct from the container for selectively closing the opening.

### BRIEF DESCRIPTION OF THE FIGURES

The present invention will be clearly understood from the description that follows with references the appended schematic figures showing, as non-limiting examples, two exemplary embodiments of this packaging device.

FIG. 1 is a perspective view of a packaging device according to a first embodiment.

FIG. 2 is a perspective view of the packaging device in FIG. 1 in which sampling tubes and a sampling bag are stored.

FIG. 3 is a perspective view of a packaging device according to a second embodiment.

FIG. 4 is a plan view, in the unfolded state, of the storage element of the packaging device in FIG. 3.

FIG. 5 is a view of the packaging device in FIG. 2 in which sampling tubes and their sampling bag are stored.

FIG. 6 is a plan view of a third possible embodiment of the packaging device according to the invention with the cover in the open position with regard to the container.

FIG. 7 is a view similar to FIG. 6 with a cover in the closed position with regard to the container.

FIG. 8 is a perspective view of the back of the container in FIG. 7 in the open position.

FIG. 9 is a front view of the open front of the container in FIG. 7.

FIG. 10 is a perspective view of the back of the cover in FIG. 6 or 7 in the open position.

### DETAILED DESCRIPTION OF THE INVENTION

The packaging device according to the invention serves to package at least two types of different receptacles, such as the packaging of sampling tubes and bags for example. This arrangement serves to identify a large number of receptacles by apposing data only on the outer surface of the container of the packaging device. This results in a reduction of the operating time and of the risks of error in the identification of the various receptacles.

Furthermore, this arrangement serves to gather together, in a single location, the various receptacles pertaining to the same patient, thereby facilitating the management of all the receptacles. Preferably, the two storage zones of the first and second types of receptacle are separated from one another by separating means.

According to a first embodiment of the invention, the container comprises a separating wall designed to divide the container into at least first and second recesses, the first recess for accommodating the storage element and the second recess forming the storage zone for the second type of receptacle of biological products.

Advantageously, the separating element or elements comprise a separating wall extending in the longitudinal direction along substantially the whole length of the container.

According to a second embodiment of the invention, the storage element comprises a first storage zone for the first type of receptacle of biological products and a second storage zone for the second type of receptacle of biological products.

Advantageously, the second storage zone is bounded by a recess arranged in the storage element (itself).

Preferably, the storage element is made in one piece and obtained by bending a sheet comprising a central portion, a first side portion joined to the central portion by a first intermediate portion, and a second side portion joined to the central portion by a second intermediate portion, the second storage zone being bounded, after bending of the sheet, by the central portion, the first side portion and the two intermediate portions.

According to another feature of the invention, the first storage zone is bounded, after bending of the sheet, by the first and second side portions, the second side portion comprising at least one row of openings for forming means for securing the first type of receptacle.

According to one feature of the invention, the storage zone for the second type of receptacle of biological products is designed to accommodate a bag for sampling biological products.

According to another feature of the invention, the storage element comprises members for securing the first type of receptacle of biological products, these securing members being arranged to accommodate at least one tubular receptacle, such as a tube for sampling biological products.

Advantageously, the container is a box having a substantially parallelepiped shape comprising opening means.

According to other possible features:
the cover comprises a cap having the shape of a tube closed at one of its ends and shaped to selectively cover the opening and a portion of the container in the longitudinal direction,
the container has the general shape of a tube closed at one of its ends, the end faces of the container each having a surface area lower than or equal to the area of each of the other faces of the container,
the edge of the open side of the cap is bevelled with respect to the perpendicular axis to the direction of introduction into or removal from the container,
at least part of the separating wall projects from one of the faces of the container in the longitudinal direction at the opening,
the cap has a cross section matching the cross section of the container,
the container and/or the cap comprises orifices distributed along the longitudinal direction, preferably close to the side faces for circulating cryogenic fluid in the device.

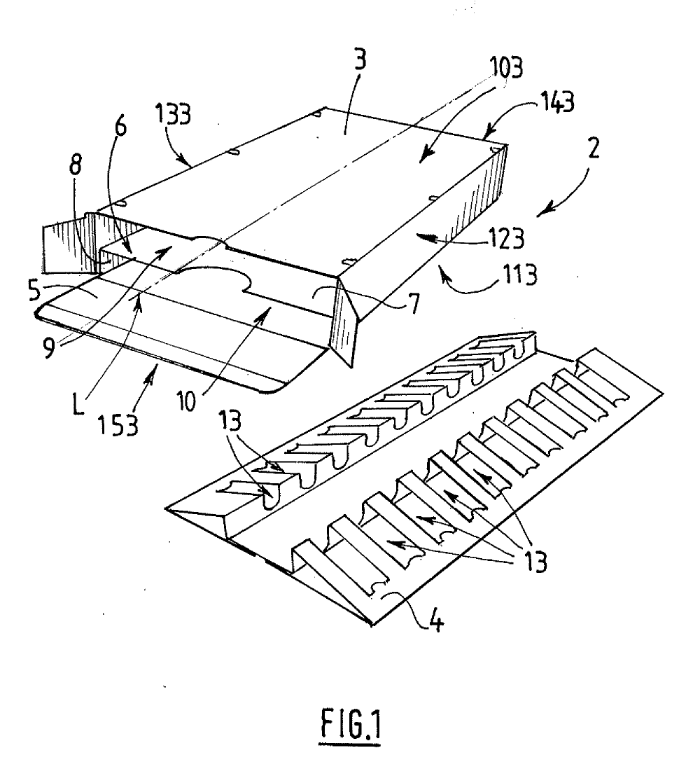

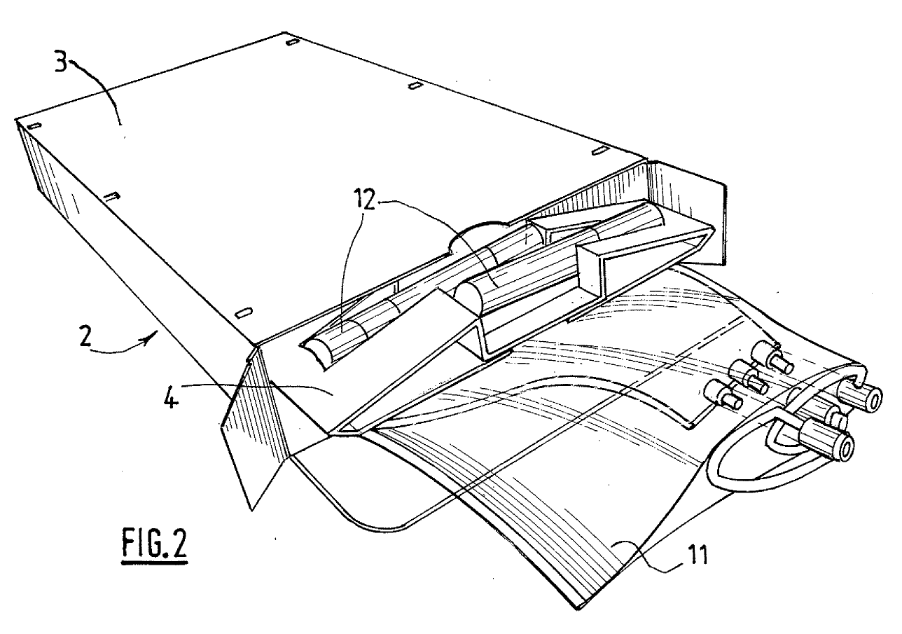

For a further understanding of the nature and objects for the present invention, reference should be made to the detailed description, taken in conjunction with the accompanying figures, in which like elements are given the same or analogous reference numbers. FIGS. 1 and 2 show a packaging device 2 for biological products, made of cardboard for example. The packaging device 2 comprises a container 3 which accommodates a storage element 4 for tubes for sampling biological products.

The container 3 consists of a parallelepiped-shaped box. The box comprises an opening cover 5 consisting of one face of the box having a flap and a hinge-forming zone.

The container 3 comprises a separating wall 6 placed inside the box and extending along at least part of the length of the box. The separating wall 6 comprises for example a central portion 7 extending parallel to the upper and lower faces of the box, and two side portions 8 extending substantially parallel to the side faces of the box. The free longitudinal edges of the side portions 8 bear against the lower longitudinal edges of the box.

The separating wall 6 is designed to divide the box into first and second recesses 9, 10 for example substantially identical.

As shown in FIG. 2, the first recess 9 is suitable for accommodating the storage element 4 while the second recess 10 forms a storage zone for a bag 11 for sampling biological products.

The storage element 4 is made in one piece and has a substantially rectangular shape. It should be observed that the length and width of the storage element 4 are substantially identical to those of the container 3.

The storage element 4 comprises means for securing sampling tubes 12 of biological products.

These securing means comprise first and second opposite rows of recesses 13 for receiving sampling tubes 12. The recesses 13 of each row are identical and uniformly spaced along the storage element 4. The recesses 13 extend perpendicular to the direction for inserting the storage element 4 into the recess 9 arranged in the container 3.

It should be observed that the two rows of recesses 13 are longitudinally offset so that a recess 13 belonging to one row is not positioned opposite one of the recesses 13 of the other row.

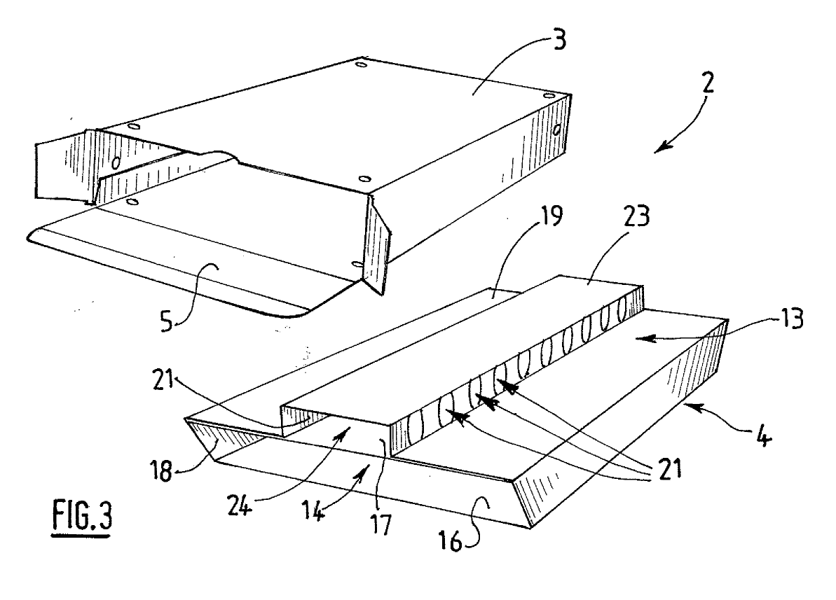

FIG. 3 shows a second embodiment of the packaging device 2 which differs from the one shown in FIG. 1 essentially in that the container 3 does not comprise a separating wall and in that the storage element 4 comprises a first storage zone 13 for sampling tubes of biological products and a second storage zone 14 for a sampling bag of biological products.

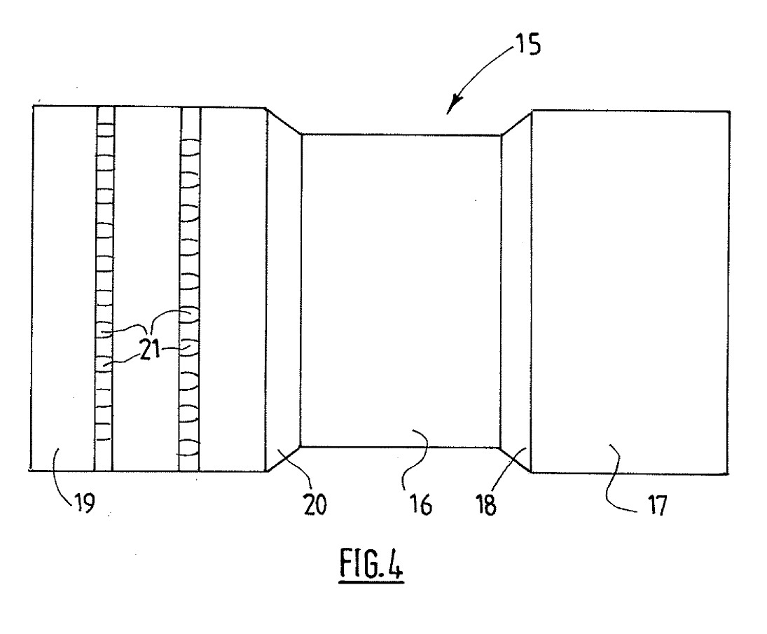

According to this embodiment, the storage element 4 is made in one piece and obtained by bending a cardboard sheet 15 shown in FIG. 4.

The cardboard sheet 15 comprises a rectangular central portion 16, a first rectangular side portion 17 joined to the central portion 16 by a first trapezoidal portion 18, and a second rectangular side portion 19 joined to the central portion 16 by a second trapezoidal portion 20.

It should be observed that the portions 16 and 17 have a width substantially equal to that of the container 3.

The second side portion 19 comprises the first and second parallel rows of identical and substantially circular openings 21. The openings 21 of each row are uniformly spaced along the second side portion 19.

It should be observed that the openings 21 belonging to one row are longitudinally offset from the openings 21 of the other row.

As shown in FIGS. 3 and 5, the sheet 15 is designed to bound, after bending, on the one hand the lower recess having a substantially rectangular cross section constituting the second storage zone 14 and suitable for accommodating a sampling bag 22 of biological products, and on the other, a central shoulder 23 placed above the second storage zone 14 and bounding a central recess 24 having a substantially rectangular cross section and suitable for accommodating sampling tubes 25 of biological products.

As shown in FIG. 3, the second storage zone 14 is bounded by the central portion 16, the side portion 17 and the two trapezoidal portions 18, 20 of the sheet 15, while the first storage zone 13 is bounded by the side portions 17 and 19 of the sheet 15.

The side portion 19 is bent so that, with the side portion 17, it forms the central recess 24 placed above the second storage zone 14.

As shown in FIG. 5, in service conditions, the openings 21 arranged on the side portion 19 constitute means for securing sampling tubes 25 of reagent products.

Each opening 21 allows the insertion of part of a sampling tube 25 into the recess 24 in a direction perpendicular to the direction of insertion of the storage element 4 into the container 3.

It should be observed that the two storage zones 13, 14 are separated from one another by the side portion 17 which thereby forms a separating wall.

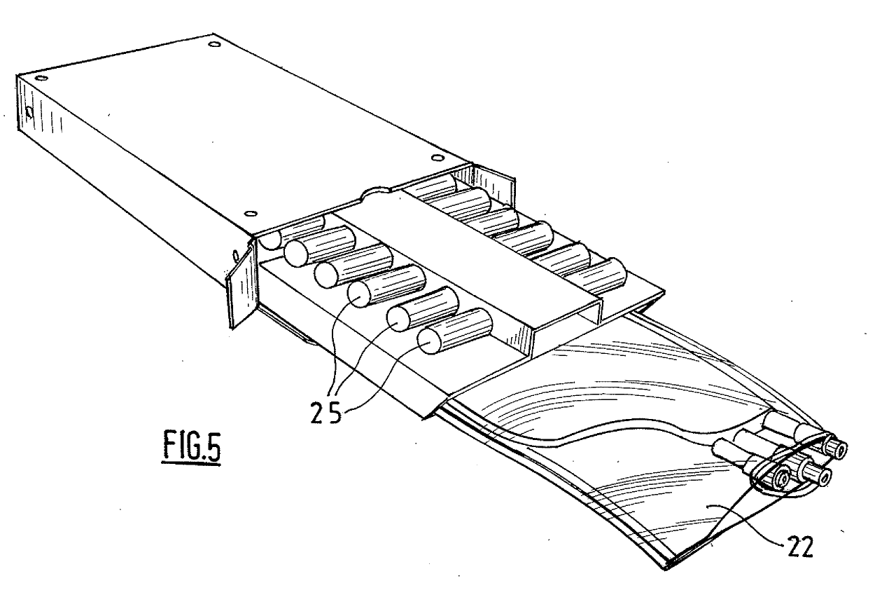

FIGS. 6 to 10 show another possible embodiment of the invention.

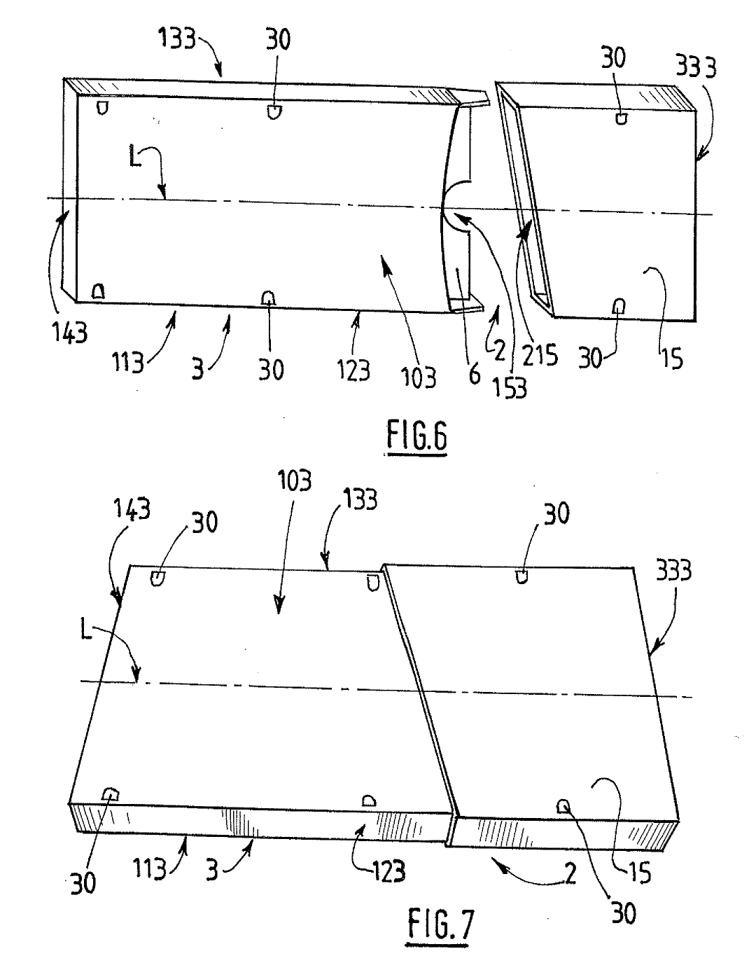

The alternative in FIGS. 6 to 10 is distinguished from that in FIG. 1 in that the opening of the container 3 (parallelepiped-shaped box) is reclosable via a distinct cap 15 suitable for being slipped on one end of the container 3 (FIG. 7) or being separated (FIG. 6) to permit the introduction/removal of the storage element 4 and/or of a sampling bag 11 for example.

As shown, the open edge 215 of the cap 15 may be bevelled with regard to the axis perpendicular to the longitudinal direction (L) of the box to facilitate its positioning on the box 3 (for this purpose the cap 15 has a shape matching that of the box).

Similarly, the box 3 and/or the cap 15 may comprise orifices 30 distributed along the length for the entry of cryogenic liquid when the container is stored in a cryogenic reservoir, and also to permit its removal (outflow) when the container is withdrawn by a user. These orifices serve to maintain the stored products at a suitable and uniform temperature.

Inscriptions may be formed on all or part of the faces 103, 113, 123, 133, 143 of the container 3 and/or of the cap 15 (definition and reference of the products stored in each recess for example).

The separating wall 6 of the container may project from the side of the opening 153 and may also comprise a cutout for better manual gripping of the objects in each of the two recesses 9, 10.

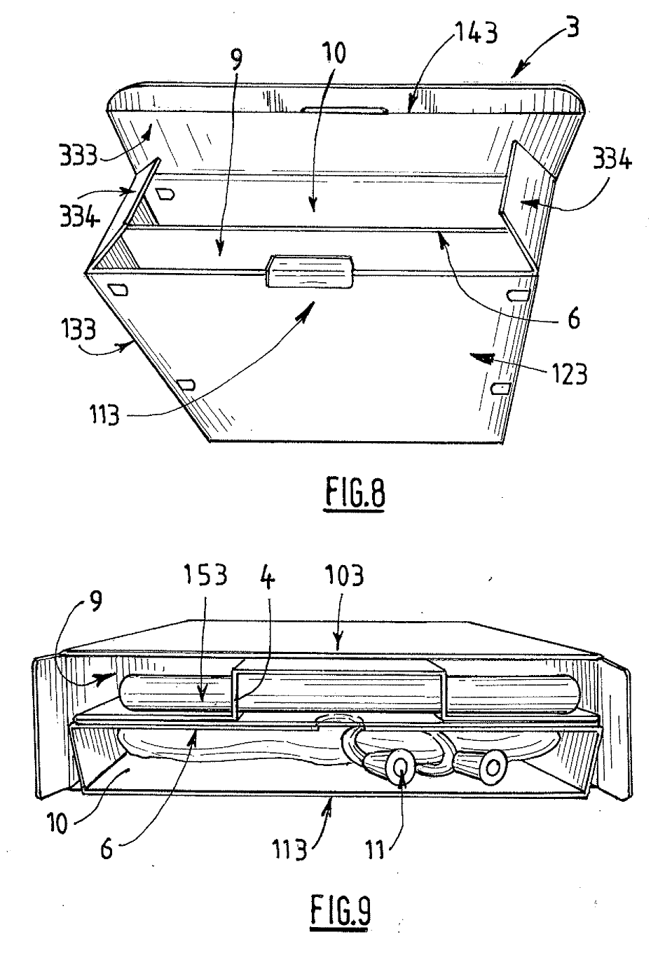

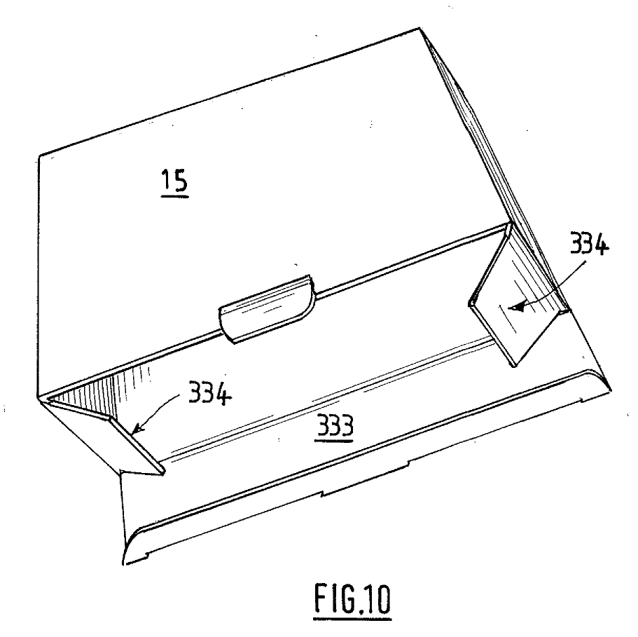

As shown in FIGS. 8 and 10, the container 3 and/or the cover 15 may be made from one or more bent sheets.

For example, the closed end of the container 153 and/or the cap 15 may be obtained by one of more flaps 333, 334 joined to one face via a hinge-forming zone. In other words, the closed end faces of the container 253, respectively of the cover 333, may be reclosable selectively via flaps joined to the container (respectively to the cover 15).

As it may be understood per se, the invention is not limited to the embodiments of this packaging device described above as examples, and on the contrary, it encompasses all alternative embodiments.

It will be understood that many additional changes in the details, materials, steps and arrangement of parts, which have been herein described in order to explain the nature of the invention, may be made by those skilled in the art within the principle and scope of the invention as expressed in the appended claims. Thus, the present invention is not intended to be limited to the specific embodiments in the examples given above.

What is claimed is:

1. A packaging device for biological products, comprising a parallelepiped-shaped container having an upper face, a lower face, and two side faces extending in a longitudinal direction between two end faces and defining at least one first recess, the device comprising a distinct storage element to be housed immovably in the first recess of the container in a direction parallel to the longitudinal direction, the storage element comprising a storage zone for a first type of receptacle of biological products, the storage element and/or the container being conformed to bound at least one second recess for a second type of receptacle of biological products, the container comprising an opening on an end face provided for introducing or removing the storage element and/or the second type of receptacle, the device comprising a cover distinct from the container for selectively closing the opening.

2. The packaging device of claim 1, wherein the two storage zones of the first and second types of receptacle are separated from one another by at least one separating element.

3. The packaging device of claim 1, wherein the container comprises a separating wall designed to divide the container into at least first and second recesses, the first recess for accommodating the storage element and the second recess forming the storage zone for the second type of receptacle of biological products.

4. The packaging device of claim 3, wherein the separating wall extends in the longitudinal direction along substantially the whole length of the container.

5. The packaging device of claim 1, wherein the storage element comprises a first storage zone for the first type of receptacle of biological products and a second storage zone for the second type of receptacle of biological products.

6. The packaging device of claim 5, wherein the second storage zone is bounded by a recess arranged in the storage element.

7. The packaging device of claim 5, wherein the storage element is made in one piece and obtained by bending a sheet comprising a central portion, a first side portion joined to the central portion by a first intermediate portion, and a second side portion joined to the central portion by a second intermediate portion, the second storage zone being bounded, after bending of the sheet, by the central portion, the first side portion and the two intermediate portions.

8. The packaging device of claim 7, wherein the first storage zone is bounded, after bending of the sheet, by the first and second side portions, and in that the second side portion comprises at least one row of openings for forming means for securing the first type of receptacle.

9. The packaging device of claim 1, wherein the storage zone for the second type of receptacle of biological products is designed to accommodate a bag for sampling biological products.

10. The packaging device of claim 1, wherein the storage element comprises members for securing the first type of receptacle of biological products, these securing members being arranged to accommodate at least one tubular receptacle, such as a tube for sampling biological products.

11. The packaging device of claim 1, wherein the cover comprises a cap having the shape of a tube closed at one of its ends and shaped to selectively cover the opening and a portion of the container in the longitudinal direction.

12. The packaging device of claim 1, wherein the container has the general shape of a tube closed at one of its ends, the end faces of the container each having a surface area lower than or equal to the area of each of the other faces of the container.

13. The packaging device of claim 1, wherein at least part of the separating wall projects from one of the faces of the container in the longitudinal direction at the opening.

14. The packaging device of claim 1, wherein the container and/or the cap comprises orifices distributed along the longitudinal direction for circulating cryogenic fluid in the device.

15. The packaging device of claim 14, wherein the orifices distributed along the longitudinal direction are close to the faces.

16. The packaging device of claim 2, wherein the container comprises a separating wall designed to divide the container into at least first and second recesses, the first recess for accommodating the storage element and the second recess forming the storage zone for the second type of receptacle of biological products.

17. The packaging device of claim 16, wherein the separating wall extends in the longitudinal direction along substantially the whole length of the container.

18. The packaging device of claim 6, wherein the storage element is made in one piece and obtained by bending a sheet comprising a central portion, a first side portion joined to the central portion by a first intermediate portion, and a second side portion joined to the central portion by a second intermediate portion, the second storage zone being bounded, after bending of the sheet, by the central portion, the first side portion and the two intermediate portions.

19. The packaging device of claim 18, wherein the first storage zone is bounded, after bending of the sheet, by the first and second side portions, and in that the second side portion comprises at least one row of openings for forming means for securing the first type of receptacle.

20. The packaging device of claim 19, wherein the storage zone for the second type of receptacle of biological products is designed to accommodate a bag for sampling biological products.

21. The packaging device of claim 20, wherein the storage element comprises members for securing the first type of receptacle of biological products, these securing members being arranged to accommodate at least one tubular receptacle, such as a tube for sampling biological products.

22. The packaging device of claim 21, wherein the cover comprises a cap having the shape of a tube closed at one of its ends and shaped to selectively cover the opening and a portion of the container in the longitudinal direction.

23. The packaging device of claim 22, wherein the container has the general shape of a tube closed at one of its ends, the end faces of the container each having a surface area lower than or equal to the area of each of the other aces of the container.

24. The packaging device of claim 23, wherein at least part of the separating wall projects from one of the faces of the container in the longitudinal direction at the opening.

25. The packaging device of claim 24, wherein the container and/or the cap comprises orifices distributed along the longitudinal direction for circulating cryogenic fluid in the device.

26. A method for storing two distinct types of biological products, said method comprising:
a. introducing a first type of receptacle of biological products and/or a second type of receptacle of biological products into a packaging device comprising a parallelepiped-shaped container that has an upper face, a lower face, and two side faces extending in a longitudinal direction between two end faces and defining at least one first recess, the device comprising a distinct storage element to be housed immovably in the first recess of the container in a direction parallel to the longitudinal direction, the storage element comprising a storage zone for the first type of receptacle of biological products, the storage element and/or the container being conformed to bound at least one second recess for the second type of receptacle of biological products, the container comprising an opening on an end face provided for introducing or removing the storage element and/or the second type of receptacle, the device further comprising a cover distinct from the container for selectively closing the opening; and
b. closing said opening of the device with the cover.
